(19) United States
(12) Patent Application Publication
(10) Pub. No.: US 2023/0052610 A1
(43) Pub. Date: Feb. 16, 2023

(54) CANNABIS PLANTS HAVING A DISTINCT CANNABINOID PROFILE

(71) Applicant: Anewsha Holding Group LLC, San Juan, PR (US)

(72) Inventors: Chris Thomas Swartout, East Marion, NY (US); Peter Horst Behncke, Jackson, MI (US)

(21) Appl. No.: 17/706,486

(22) Filed: Mar. 28, 2022

Related U.S. Application Data
(63) Continuation of application No. 16/460,952, filed on Jul. 2, 2019, now abandoned.

(51) Int. Cl. A01H 6/28; A01H 5/12

(52) U.S. Cl. CPC A01H 6/28; A01H 5/12

(57) ABSTRACT

The disclosure relates to Cannabis plants having a distinct cannabinoid profile and limited psychoactive properties that can be suitable for medicinal uses associated with treating inflammation and chronic pain.

### CROSS-REFERENCE TO RELATED APPLICATIONS

This application is a continuation of U.S. patent application Ser. No. 16/460,952, filed Jul. 2, 2019, which claims priority to U.S. Provisional Patent Application No. 62/710,868, filed May 2, 2018 the disclosure of each of which are hereby incorporated by reference in their entireties.

### BACKGROUND OF DISCLOSURE

### Field

The present disclosure provides Cannabis plants having a distinct cannabinoid profile and limited psychoactive properties that can be suitable for medicinal uses associated with treating inflammation and chronic pain.

### Technical Background

Cannabis is a highly variable genus of flowering plant that includes at least three species: Cannabis sativa, Cannabis indica, and Cannabis ruderalis. (U.S. PP27475P2, incorporated herein by reference). There are hundreds of different biologically active chemicals known to exist in Cannabis plants (phytochemicals). For medical Cannabis plants, cannabinoids and terpenes are the key phytochemicals to harness medicinal properties. Two cannabinoids that can be produced in high abundance from Cannabis plants are cannabidiol (CBD) and Δ9-tetrahydrocannabinol (THC).

THC is the primary psychoactive cannabinoid and is the result of the decarboxylation of tetrahydrocannabinolic acid (THC-A), its acidic precursor. THC-A, (6ar,10ar)-1-hydroxy-6,6,9-trimethyl-3-pentyl-6a,7,8,10a-tetrahydro-6h-benzochromene-2-carboxylic acid, is found in the trichomes of the plant and converted into THC, which exists in only minute quantities in the living plant, after harvest and drying. Cannabigerol (CBG), Resorcinol, 2-(3,7-dimethyl-2,6-octadienyl)-5-pentyl-, is not considered psychoactive, is known to block the psychoactive effects of THC. Cannabichromene (CBC) and cannabidiol (CBD) are both non-psychoactive and end products of CBG metabolism. CBD and CBC are also used medicinally. CBD, THC, and other phytocannabinoids act on cannabinoid receptors located throughout the body of mammals, birds, fish, and reptiles making cannabinoids (and the Cannabis plant) suited for medicinal uses.

Thus, there is a need for new cultivars of Cannabis that are particularly suited for medical use. Described herein is a unique Cannabis plant named R2 Sativa that has medicinal properties from higher levels of CBD and no detectable levels of tetrahydrocannabivarin (THC-V), as well as a cannabinoid profile that can help with inflammation and chronic pain.

### BRIEF SUMMARY OF DISCLOSURE

The present disclosure provides Cannabis plants with unique cannabinoid profiles. One aspect of the disclosure provides a Cannabis plant or plant part comprising a cannabidiol (CBD) content of greater than about 7.5 mass % and having no detectable levels of tetrahydrocannabivarin (THC-V). In some embodiments, the CBD content is about 8-10 mass or about 9.0-9.5 mass %. In some embodiments, the cannabinoid profile further comprises tetrahydrocannabinol (Δ9-THC), wherein the Δ9-THC content is about 4.0-7.0 mass % or about 6.5-7 mass %.

In some embodiments, the cannabinoid profile further comprises one or more of cannabinol (CBN), cannabichromene (CBC), or cannabigerol (CBG), wherein the CBN content is less than about 1.0 mass %, the CBC content is about 0.5-2.0 mass %, and the CBG content is less than about 1.0 mass %. In other embodiments, the cannabinoid profile contains no detectable levels of Δ8-tetrahydrocannabinol (Δ8-THC) and cannabidivarin (CBD-V).

In other embodiments, the cannabinoid profile comprises a ratio of CBD to Δ9-THC of about 1.2:1 to about 1.5:1.

In some embodiments, the Cannabis plant or plant part of the disclosure further comprises a terpene profile comprising limonene. In some embodiments, the cannabinoid profile and/or the terpene profile can be measured using gas chromatography with a flame ionization detector (GC-FID).

In some embodiments, the Cannabis plant or plant part of the disclosure has limited psychoactive properties and is suitable for medicinal use.

In other embodiments, the Cannabis plant or plant part of the disclosure is propagated by asexual reproduction.

Another aspect of the disclosure provides a Cannabis plant or plant part comprising a cannabinoid profile having: a CBD content that is at least about 9.0 mass %; a Δ9-THC content that is less than about 7.0 mass %; a CBN content that is less than about 1.0 mass %; a CBC content that is less than about 2.0 mass %; a CBG content that is less than about 1.0 mass %; and undetectable levels of one or more of Δ8-THC, THC-V, or CBV-V, wherein the cannabinoid profile is measured using GC-FID.

Additional features and advantages are described herein, and will be apparent from the Detailed Description and the Claims.

### DETAILED DESCRIPTION

For the purposes of promoting an understanding of the principles of the present disclosure, reference will now be made to specific embodiments and specific language will be used to describe the same.

Unless otherwise defined, all terms (including technical and scientific terms) used herein have the same meaning as commonly understood by one having ordinary skill in the art to which this disclosure belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

It will be further understood that a number of aspects and embodiments are disclosed. Each of these has individual benefit and each can also be used in conjunction with one or more, or in some cases all, of the other disclosed aspects and embodiments, whether specifically delineated or not. Accordingly, for the sake of clarity, this description will refrain from repeating every possible combination of the individual aspects and embodiments in an unnecessary fashion. Nevertheless, the specification and claims should be read with the understanding that such combinations are implicitly disclosed, and are entirely within the scope of the disclosure and the claims, unless otherwise specified.

The inventors have discovered a new Cannabis plant referred to as R2 Sativa having a laboratory name of Sample 1712SPT0367.1113. This new plant is a variety of a cross between Cannabis sativa; ssp. Sativa and Cannabis sativa ssp. Indica plants. Surprisingly, R2 Sativa has a cannabinoid profile comprising: a greater CBD content relative to Δ9-THC, a variety of non-psychoactive cannabinoids (CBD, CBN, CBG, and CBC), an absence of THCV, Δ8-THC, and CBV, as well as the presence of the terpene, limonene. R2 Sativa's limited psychoactive properties make it suited for medicinal use to treat, prevent, or reduce inflammation and chronic pain.

Definitions:

Articles “a” and “an” are used herein to refer to one or to more than one (i.e. at least one) of the grammatical object of the article. By way of example, “an element” means at least one element and can include more than one element.

As used herein, the term “and/or” includes any and all combinations of one or more of the associated listed items. It will be further understood that the use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a mass percentage range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “about” in association with a numerical value means that the numerical value can vary plus or minus by 5% or less of the numerical value.

One aspect of the disclosure provides Cannabis plants or plant parts having distinct cannabinoid profiles. The term “plant” as used herein refers to plants in the genus of Cannabis (e.g., Cannabis sativa, Cannabis indica, and Cannabis ruderalis) and plants derived thereof. Different Cannabis species can be identified by both phenotypic characteristics and by chemotype (e.g., cannabinoid and terpene profiles). The Cannabis plants of the disclosure can be propagated or produced by asexual reproduction or by seed. Examples of the Cannabis plants or plant parts of the disclosure include the R2 Sativa cultivar that was discovered by the inventors.

The term “plant part” as used herein refers to any part of a plant including but not limited to the embryo, shoot, root, stem, seed, stipule, leaf, petal, flower bud, flower, ovule, bract, trichome, branch, petiole, pistil, node, internode, bark, pubescence, tiller, rhizome, frond, blade, fan leave, sugar leave, cola, calyx, ovule, pollen, stamen, and the like.

As used herein, the term “cultivar” means a group of similar plants that by structural features and performance (i.e., morphological and physiological characteristics) can be identified from other varieties within the same species. Furthermore, the term “cultivar” refers to a variety, strain or race of plant that has been produced by horticultural or agronomic techniques and is not found in natural or wild populations. The terms “cultivar,” “variety,” “strain” and race are often used interchangeably by plant breeders, agronomists, and farmers, and are used interchangeably herein.

The term “cannabinoid profile” as used herein refers to the mixture of cannabinoids produced by a Cannabis plant or plant part. Selective breeding can be used to control the genetics of Cannabis plants and change the cannabinoid and/or terpene profiles. For example, cannabis cultivars used for medicinal purposes can have higher CBD content and lower THC content to reduce or limit the psychoactive properties of the cultivar.

The term “cannabinoid” as used herein means any chemical substance that acts upon a cannabinoid receptor. For example the term cannabinoid includes, but is not limited to, cannabinoid ligands such as agonists, partial agonists, inverse agonists, or antagonists, as demonstrated by binding studies and functional assays. A cannabinoid can be identified because its chemical name will include “cannabi” in the name. Where reference is made to a particular cannabinoid herein, each of the acid and/or decarboxylated forms are contemplated as both single molecules and mixtures.

Examples of cannabinoids include, but are not limited to, compounds belonging to any of the following classes of molecules, their derivatives, salts, or analogs: Tetrahydrocannabinol (Δ9-THC), Δ8-Tetrahydrocannabinol (Δ8-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), cannabidivarin (CBDV), Δ9-Tetrahydrocannabivarin (THCV), cannabichromevarin (CBCV), cannabigerovarin (CBGV), and isocanabinoids. Examples of non-psychoactive cannabinoids include, but are not limited to, CBD, CBC, CBE, CBG, CBN, CBND, CBT, CBDV, CBGV, and CBCV.

The term “terpene profile” as used herein refers to the mixture of terpenes produced by a Cannabis plant or plant part.

The term “terpene” as used herein refers to an organic compound that is derived biosynthetically from units of isopentenyl pyrophosphate. Terpene molecules found in plants can be the primary constituents of essential oils and can produce fragrances and smells. Terpenes can be monoterpenoids, sesquiterpenoids, sesterterpenoid, sesquarterpenoids, tetraterpenoids, Triterpenoids, tetraterpenoids, Polyterpenoids, isoprenoids, and steroids. Terpenes can be: α-, β-, γ-, oxo-, isomers, or combinations thereof. Examples of terpenes include, but are not limited to, 8-dihydroionone, Acetanisole, Acetic Acid, Acetyl Cedrene, Anethole, Anisole, Benzaldehyde, Bergamotene (γ-cis-Bergamotene, α-trans-Bergamotene), Bisabolol (β-Bisabolol, α-Bisabolol), Borneol, Bornyl Acetate, Butanoic/Butyric Acid, Cadinene (α-Cadinene, γ-Cadinene), Cafestol, Caffeic acid, Camphene, Camphor, Capsaicin, Carene (A-3-Carene, delta-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene, β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene, γ-Curcumene), Decanal, Dehydrovomifoliol, Diallyl Disulfide, Dihydroactinidiolide, Dimethyl Disulfide, Eicosane/Icosane, Elemene (8-Elemene), Estragole, Ethyl acetate, Ethyl Cinnamate, Ethyl maltol, Eucalyptol/1,8-Cineole, Eudesmol (α-Eudesmol, β-Eudesmol, γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10),11-diene, Guaiacol, Guaiene (α-Guaiene), Gurjunene (α-Gurjunene), Herniarin, Hexanaldehyde, Hexanoic Acid, Humulene (α-Humulene, β-Humulene), Ionol (3-oxo-α-ionol, 13-Ionol), Ionone (α-Ionone, β-Ionone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (p-Myrcene, β-Myrcene), γ-Muurolene, Nepetalactone, Nerol, Nerolidol, trans-Nerolido, Neryl acetate, Nonanaldehyde, Nonanoic Acid, Ocimene, Octanel, Octanoic Acid, P-cymene, Pentyl butyrate, Phellandrene, Phenylacetaldehyde, Phenylethanethiol, Phenylacetic Acid, Phytol, Pinene (α-Pinene, β-Pinene), Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpinene, γ-Terpinene, Terpinolene, Thiophenol, Thujone, Thymol, α-Tocopherol, Tonka Undecanone, Undecanal, Valeraldehyde/Pentanal, Verdoxan, α-Ylangene, or Umbelliferone.

Quantitative analysis of a plant's cannabinoid profile or terpene profile can be determined by gas chromatography (GC), including but not limited to, gas chromatography combined with mass spectrometry (GC/MS) or gas chromatography combined with a flame ionization detector (GC-FID). The majority of THC and CBD exist naturally as tetrahydrocannabinol acid (THCA) and cannabidiol acid (CBDA) within the plant. These molecules undergo decarboxylation to form THC and CBD when heated (e.g., consumed by smoking, cooked, or vaporized within a gas chromatography machine).

Liquid chromatography (LC) techniques (e.g., ultra performance liquid chromatography UPLC) are also possible and, unlike GC methods, can differentiate between the acid and neutral forms of the cannabinoids because LC does not vaporize the samples.

The term “no detectable levels” of a particular cannabinoid (e.g., THC-V, Δ8-THC, or CBDV) can be determined using GC or LC techniques that are known in the art.

As used herein, the term “mass percent (%)” in reference to cannabinoid content or terpene content is one way of representing the concentration of a cannabinoid or terpene in a plant. Mass percent refers to the percentage of the plant's total mass that is comprised of that particular cannabinoid or terpene. For example, a plant having a total mass of 100 grams, comprising 50 total milligrams of CBD, would have a CBD content of 50 mass %. As another example, it would be appreciated that if a Cannabis plant of the disclosure had a CBD content of 100 mg/g that the corresponding mass % would be 10 mass %.

In some embodiments, the Cannabis plant or plant part has a cannabinoid profile comprising, for example, Δ9-THC, CBD, CBN, CBC, CBG, and non-detectable levels of Δ8-THC, THCV, and CBDV. This cannabinoid profile can cause the Cannabis plant or plant part to have limited psychoactive properties and be suitable for medicinal uses for chronic pain and inflammation. The term “limited psychoactive properties” as used herein refers to a milder psychoactive effect in the subject (e.g., a human or animal) when the Cannabis plant or plant part of the disclosure are consumed as compared to a Cannabis plant or plant part having higher levels of Δ9-THC or other psychoactive cannabinoids.

In some embodiments, the Cannabis plant or plant part has a cannabinoid profile comprising a CBD content of greater than about 7.5 mass % (e.g., 7.6 mass %, 7.8 mass %, 8.0 mass %, 8.5 mass %, 9.0 mass %, 9.5 mass %, 10.0 mass %, or 10.5 mass %). In other embodiments, the CBD content is about 8.0-10.0 mass % (e.g., 7.8 mass %, 7.9 mass %, 8.0 mass %, 8.1 mass %, 8.2 mass %, 8.3 mass %, 8.4 mass %, 8.5 mass %, 8.6, mass %, 8.7 mass %, 8.8 mass %, 8.9 mass %, 9.0 mass %, 9.1 mass %, 9.2 mass %, 9.3 mass %, 9.4 mass %, 9.5 mass %, 9.6 mass %, 9.7 mass %, 9.8 mass %, 9.9 mass %, 10 mass %, 10.1 mass % 10.2 mass %, 10.3 mass %, 10.4 mass %, or 10.5 mass %). In other embodiments, the CBD content of the Cannabis plant or plant part is about 9.0-9.5 mass %.

In some embodiments, the Cannabis plant or plant part has a cannabinoid profile comprising Δ9-THC, wherein the Δ9-THC content is about 4.0-7.0 mass % (e.g., about 3.8 mass %, 3.9 mass %, 4.0 mass %, 4.1 mass %, 4.2 mass %, 4.3 mass %, 4.4 mass %, 4.5 mass %, 4.6 mass %, 4.7 mass %, 4.8 mass %, 4.9 mass %, 5.0 mass %, 5.1 mass %, 5.2 mass %, 5.3 mass %, 5.4 mass %, 5.5 mass %, 5.6 mass %, 5.7 mass %, 5.8 mass %, 5.9 mass %, 6.0 mass %, 6.1 mass %, 6.2 mass %, 6.3 mass %, 6.4 mass %, 6.5 mass %, 6.6 mass %, 6.7 mass %, 6.8 mass %, 6.9 mass %, 7.0 mass %, 7.1 mass %, 7.2 mass %, 7.3 mass %). In other embodiments, the Δ9-THC content is about 6.5-7.0 mass %.

In some embodiments, the Cannabis plant or plant part described herein has a cannabinoid profile comprising a CBN content that is less than about 1 mass % (e.g., about 0.9 mass %, 0.8 mass %, 0.7 mass %, 0.6 mass %, 0.5 mass %, 0.4 mass %, 0.3 mass %, 0.2 mass %, 0.1 mass %), a CBC content that is about 0.5-2 mass % (e.g., about 0.3 mass %, 0.4 mass %, 0.5 mass %, 0.6 mass %, 0.7 mass %, 0.8 mass %, 0.9 mass %, 1.0 mass %, 1.1 mass %, 1.2 mass %, 1.3 mass %, 1.4 mass %, 1.5 mass %, 1.6 mass %, 1.7 mass %, 1.8 mass %, 1.9 mass %, 2.0 mass %, 2.1 mass %, 2.2 mass %), and the CBG content is less than about 1 mass % (e.g., about 0.9 mass %, 0.8 mass %, 0.7 mass %, 0.6 mass %, 0.5 mass %, 0.4 mass %, 0.3 mass %, 0.2 mass %, 0.1 mass %).

In other embodiments, the cannabinoid profile of the Cannabis plant or plant part described herein comprises a ratio of CBD to Δ9-THC of about 1.2:1 to about 1.5:1 (e.g., about 1.1:1, 1.2:1, 1.3:1, 1.4:1, 1.5:1, 1.6:1, or 1.7:1).

In other embodiments, the Cannabis plant or plant part comprises a cannabinoid profile having: a CBD content that is at least about 9 mass %; a Δ9-THC content that is less than about 7 mass %; a CBN content that is less than about 1 mass %; a CBC content that is less than about 2 mass %; a CBG content that is less than about 1 mass %; and undetectable levels of one or more of Δ8-THC, THC-V, or CBV-V, wherein the cannabinoid profile is measured using GC-FID.

In yet other embodiments, the Cannabis plant or plant part of the disclosure, further comprising a terpene profile comprising limonene.

The Cannabis plant or plant part of the disclosure can be propagated by asexual reproduction by methods known in the art. Asexual propagation can be done from off-shoots of a mother plant in the vegetative state. The cloned shoots can be isolated in a humidity dome until roots form, and then the clone can be transferred to a suitable substrate.

The Cannabis plant or plant part of the disclosure can be suitable for medicinal use. As used herein, the term “suitable for medicinal use” refers to the ability to consume or topically apply the Cannabis plant or plant part in order to help relieve, treat, prevent, lessen, or ameliorate a symptom of a disease, illness, or ailment experienced in a subject. Chronic pain and inflammation are non-limiting examples of the types of ailments that the Cannabis plants and plant parts of the disclosure can help treat and prevent. A Cannabis plant or plant part that is suitable for medicinal use will also have limited psychoactive properties.

The following examples are offered by way of illustration and not by way of limitation.

### EXAMPLE 1: CULTIVATING A NEW CANNABIS PLANT TO CONTROL INFLAMMATION

To create a cultivar best suited to control inflammation, various strains of Cannabis sativa were cultivated. In particular, the plant referred to as “R2 Sativa” described herein was derived from a female said to be of a specific variety called colloquially, “Pinneapple,” an individual grown indoors and was of the typical subspecies, Cannabis sativa ssp. sativa. The male parent was a strain known colloquially as “AC DC” and reportedly to belonged to the subspecies Cannabis sativa ssp. indica (Lam.).

The particular plant disclosed herein was discovered in the area of Jackson and Calhoun County, Mich. under the legal Michigan Medical Marihuana Act where the inventors were intentionally crosspollinating and cultivating plants of cross between those described above using standard Mendelian breeding techniques. This resulted in the R2 generation of the inventors' cross, named “R2.” It was in the proximity of plants of the R2 variety that had become hermaphroditic, in the inventors' legal growing Research and Development facility in Michigan which discovered one female plant that could only be reproduced asexually, by taking cuttings, and that plant is the origin of this new cultivar. The female plant was discovered in a section under review. The plant has been and continues to be asexually reproduced by cutting at the Research and Development facility.

### EXAMPLE 2: R2 SATIVA CANNABINOID PROFILE

The cannabinoid profile for R2 Sativa was determined by laboratory testing by Spott Labs (Kalamazoo, Mich.). A sample containing 0.75 grams of cured plant flower for R2 Sativa was analyzed by gas chromatography (GC-FID). The sample was found to be free of mold, pests, or other contaminants.

The testing results, shown in Table 1, demonstrated that R2 Sativa contains cannabidiol (CBD) at 94.14 mg/g (9.414 mass %), Δ9-THC at 69.12 mg/g (6.912 mass %), CBC at 10.04 mg/g (1.004 mass %), CBG at 6.11 mg/g (0.611 mass %), cannabinol (CBN) at 3.62 mg/g (0.362 mass %), THC-V at 0.00 mg/g, Δ8-THC at 0.00 mg/g, and CBDV at 0.00 mg/g.

TABLE 1
R2 Sativa Cannabinoid Profile

| Cannabinoid | Mass % | Mass (mg/g) |
|---|---|---|
| Δ9-THC | 6.912 | 69.12 |
| CBD | 9.414 | 94.14 |
| CBN | 0.362 | 3.62 |
| CBC | 1.004 | 10.04 |
| CBG | 0.611 | 6.11 |
| Δ8-THC | Not detected | Not detected |
| THCV | Not detected | Not detected |
| CBDV | Not detected | Not detected |
| Total concentration of | 18.304 | 183.038 |
| cannabinoids* | | |
| Total THC** | 6.912 | 69.12 |
| Total CBD** | 9.414 | 94.14 |

*This total concentration also represents the cannabinoid concentration when the product is fully cooked or burned prior to consumption.
**Total THC is calculated by the formula: THCa * 0.877 + Δ9-THC; total CBD is calculated by the formula CBDa * 0.877 + CBD.
***The Limit of Quantification for the data in Table 1 was 0.002% LOQ.

R2 Sativa cannabinoid content is dominated by its high content of CBD (94.14 mg/g) (9.414% Mass) and no THC-V (0.00 mg/g) (non detected percentage Mass).

Most varieties of high potency cannabis contain large quantities of three specific terpenes as well as various assortments of others. Those three terpenes are beta-myrcene, beta-caryophyllene and linalool. R2 Sativa was found to have a unique complement of terpenes. There are specific levels of beta-myrcene, beta-caryophyllene, and linalool, but the limonene level combined with the undetectable level of THC-V is unique and different. R2 Sativa is distinct from other varieties in its cannabinoid profile and its medical properties.

There are indications that use of R2 Sativa can prevent inflammation and chronic pain. The levels of limonene can slow down the build-up of plaque in the arteries and reduce the effect of low-density lipo-proteins on the circulatory system. R2 Sativa has medical qualities that make it an important cultivar to maintain health and treat and prevent inflammation and chronic pain.

### EXAMPLE 3: R2 SATIVA CANNABINOID PROFILE COMPARED TO PLANTS WITH SIMILAR PARENTS

Comparison of the terpenoid/cannabinoid profiles of R2 Sativa with other plants with the similar parents, reveals that this plant, R2 Sativa has a phenotypically unique profile, particular insofar as its levels of limonene, THC-V, THC, CBN, and CBD, giving it a rare unique combination to treat inflammation. Two plants with the same parents, referred to as A and B, and R2 Sativa were tested. Limonene, THC-V, CBN and CBD found in R2 Sativa was statistically different than the range of A and B. R2 Sativa was shown to have substantially higher levels of cannabidiol (CBD), Cannabigerol (CBG), and (Δ9-THC) (69.12 mg/g). R2 Sativa had levels of 94.14 mg/g of CBD while A and B plants had much lower range of CBD. R-2 Sativa had levels of CBC of 10.04 mg/g while A and B had trace amounts of CBC. R2 Sativa had no detected mg/g of THC-V and 3.62 mg/g of CBN.

### EXAMPLE 4: ASEXUAL REPRODUCTION AND CULTIVATION OF R2 SATIVA

Asexual reproduction, also known as “cloning,” is a process well known to those of ordinary skill in the art of gardening and can include the following steps (as described in U.S. PP27475P2):

Step 1: From the female plant whose genetic profile is sought to be exactly reproduced, a 1-2 inch cutting is taken;

Step 2: The cutting of the desired plant and is placed in a rooting solution that may be water, or a B1 vitamin that is generally the content of a rooting or cloning powder or gel;

Step 3: The cutting and rooting solution are placed into a rooting medium such as rock wool and RAPID ROOTER® a peat moss product available at hydroponics stores, gardening supply stores, and from many Internet merchants and must be water retaining so that the roots can take water up into the leaves;

Step 4: The cutting in rooting solution and rooting medium is placed under a humidity dome that may be an inverted plastic cup or is available in kits 1 foot—2 feet long with a clear plastic dome to keep the humidity as high as possible to avoid the leaves drying before the cutting roots to insure viability;

Step 5: A fluorescent light is placed just over the humidity dome that may be a generic work light available at hardware stores;

Step 6: One to two times a day, the humidity dome is removed for 30 seconds to insure the exchange of depleted air with fresh air to replenish the CO2.

Step 7: After maintaining this cutting in this manner, keeping the rooting medium wet at all times, for 5-14 days until root formation

Step 8: The rooted plants are maintained under fluorescent high intensity lights in a soil-less medium, with standard hydroponic fertilizers, for 14 weeks;

The plants described herein can be cultivated in this manner at a temperature between 76 and 80 degrees Fahrenheit and at 14 weeks of age. The new plant differs from its parents and related cultivars in that it has limited psychoactive effects as opposed to the psychoactive effect associated with ssp. sativa.

Observation of the all-female progeny of the original plant has demonstrated that this new and distinct variety has distinctive characteristics that are firmly fixed and hold true from generation to generation propagated from the original plant.

Any patents or publications mentioned in this specification are indicative of the levels of those skilled in the art to which the disclosure pertains. These patents and publications are herein incorporated by reference to the same extent as if each individual publication was specifically and individually indicated to be incorporated by reference.

One skilled in the art will readily appreciate that the present disclosure is well adapted to carry out the objects and obtain the ends and advantages mentioned, as well as those inherent therein. The present examples along with the methods described herein are presently representative of preferred embodiments, are exemplary, and are not intended as limitations on the scope of the disclosure. Changes therein and other uses will occur to those skilled in the art which are encompassed within the spirit of the disclosure as defined by the scope of the claims.

1. A Cannabis plant or plant part having a cannabinoid profile comprising a cannabidiol (CBD) content of greater than about 7.5 mass % and having no detectable levels of tetrahydrocannabivarin (THC-V).

2. The Cannabis plant or plant part of claim 1, wherein the CBD content is about 8.0-10.0 mass %.

3. The Cannabis plant or plant part of claim 1, wherein the CBD content is about 9.0-9.5 mass %.

4. The Cannabis plant or plant part of claim 1, wherein the cannabinoid profile further comprises tetrahydrocannabinol (Δ9-THC), wherein the Δ9-THC content is about 4.0-7.0 mass %.

5. The Cannabis plant or plant part of claim 4, wherein the Δ9-THC content is about 6.5-7.0 mass %.

6. The Cannabis plant or plant part of claim 1, wherein the cannabinoid profile further comprises one or more of cannabinol (CBN), cannabichromene (CBC), or cannabigerol (CBG).

7. The Cannabis plant or plant part of claim 6, wherein the CBN content is less than about 1.0 mass %, the CBC content is about 0.5-2.0 mass %, and the CBG content is less than about 1.0 mass %.

8. The Cannabis plant or plant part of claim 1, wherein the cannabinoid profile has no detectable levels of Δ8-tetrahydrocannabinol (Δ8-THC) and cannabichromevarin (CBD-V).

9. The Cannabis plant or plant part of claim 1, wherein the cannabinoid profile comprises a ratio of CBD to Δ9-THC of about 1.2:1 to about 1.5:1.

10. The Cannabis plant or plant part of claim 1, further comprising a terpene profile comprising limonene.

11. The Cannabis plant or plant part of claim 1, wherein the plant or plant part has limited psychoactive properties.

12. The Cannabis plant or plant part of claim 1, wherein the plant or plant part is propagated by asexual reproduction.

13. The Cannabis plant or plant part of claim 1, wherein the plant or plant part is suitable for medicinal use.

14. The Cannabis plant or plant part of claim 1, wherein the cannabinoid profile is measured using gas chromatography with flame ionization detector (GC-FID).

15. The Cannabis plant or plant part of claim 10, wherein the terpene profile is measured using GC-FID.

16. A Cannabis plant or plant part comprising a cannabinoid profile having:
a) a CBD content that is at least about 9.0 mass %;
b) a Δ9-THC content that is less than about 7.0 mass %;
c) a CBN content that is less than about 1.0 mass %;
d) a CBC content that is less than about 2.0 mass %;
e) a CBG content that is less than about 1.0 mass %; and
f) undetectable levels of one or more of Δ8-THC, THC-V, or CBV-V, wherein the cannabinoid profile is measured using GC-FID.

17. The Cannabis plant or plant part of claim 16, wherein the cannabinoid profile comprises a ratio of CBD to Δ9-THC of about 1.2:1 to about 1.5:1.

18. The Cannabis plant or plant part of claim 16, wherein the plant or plant part is propagated by asexual reproduction.

19. The Cannabis plant or plant part of claim 16, further comprising a terpene profile comprising limonene.

20. The Cannabis plant or plant part of claim 19, wherein the terpene profile is measured using GC-FID.
